(19) United States
(12) Patent Application Publication
(10) Pub. No.: US 2025/0292909 A1
(43) Pub. Date: Sep. 18, 2025

(54) METHOD FOR ASSESSING ACUTE KIDNEY INJURY AND ACUTE KIDNEY INJURY ASSESSMENT SYSTEM

(71) Applicant: China Medical University, Taichung City (TW)

(72) Inventors: Chin-Chi Kuo, Taichung City (TW); Hung-Chieh Yeh, Taichung City (TW)

(73) Assignee: China Medical University, Taichung City (TW)

(21) Appl. No.: 19/222,243

(22) Filed: May 29, 2025

Related U.S. Application Data
(63) Continuation-in-part of application No. 17/105,714, filed on Nov. 27, 2020, now abandoned.

(30) Foreign Application Priority Data
Oct. 22, 2020 (TW) 109136737

(51) Int. Cl. G16H 50/20; G01N 33/493

(52) U.S. Cl. CPC G16H 50/20; G01N 33/493

(57) ABSTRACT

A method for assessing acute kidney injury includes following steps. A kidney function diagnostic dataset of a subject by a processor is obtained by a processor, wherein the kidney function diagnostic dataset includes at least one serum creatinine concentration data or at least one estimated glomerular filtration rate data of the subject. A preprocessing step is performed by the processor, wherein at least one fluctuation value of serum creatinine concentration or at least one fluctuation value of eGFR is calculated to obtain by the processor. A first analyzing step is performed by the processor to analyze the at least one fluctuation value of serum creatinine concentration or the at least one fluctuation value of eGFR by the AKI assessing model so as to define the subject is a patient with AKI or a patient without AKI.

### RELATED APPLICATIONS

This application is a Continuation-in-part of U.S. application Ser. No. 17/105,714, filed on Nov. 27, 2020, which claims priority to Taiwan Application Serial Number 109136737, filed Oct. 22, 2020, which is herein incorporated by reference.

### BACKGROUND

### Technical Field

The present disclosure relates to a medical information analysis method and a system thereof. Particularly, the present disclosure relates to a method for assessing acute kidney injury and an acute kidney injury assessment system.

### Description of Related Art

Acute kidney injury (“AKI” hereafter) is a deterioration of renal function within a short period of time which happens suddenly and can be reversible. The patient with AKI will have symptoms of abnormally increasing serum creatinine concentration and urea nitrogen. Furthermore, symptoms such as general fatigue, decreased urine output, edema, loss of appetite, nausea, vomiting, and unconsciousness may happen.

In the aspect of diagnosis, because the AKI does not have obvious indicative clinical symptoms, the main method of clinical diagnosis of the AKI is to test the changes in serum creatinine concentration by a blood draw. However, the basic condition of renal function of the patient is not always available at the first time in an outpatient clinic or hospitalization, so that not only the condition of the patient cannot be known immediately, but also the corresponding treatment will be delayed, resulting in long-term damage to the renal function. Furthermore, the current healthcare institutions do not have a medical system that can comprehensively monitor the changes in renal function of patients admitted in outpatient clinics and emergency department, which has a lot of influence on the selection and implementation of appropriate medical plans.

Therefore, how to develop a method for assessing AKI, which is automated, standardized, rapid and with high detection accuracy, is a technical issue with clinical application value.

### SUMMARY

According to one aspect of the present disclosure, a method for assessing acute kidney injury (AKI) includes following steps. A kidney function diagnostic dataset of a subject is obtained by a processor, wherein the kidney function diagnostic dataset includes at least one serum creatinine concentration data or at least one estimated glomerular filtration rate (eGFR) data of the subject, and a recording date of the at least one serum creatinine concentration data and a recording date of each of the at least one estimated glomerular filtration rate data are respectively on 0 to 180 days before an acute kidney injury assessing date of the subject. A preprocessing step is performed by the processor, wherein a changing degree over a time period of the at least one serum creatinine concentration data or a changing degree over a time period of the at least one estimated glomerular filtration rate data is calculated by the processor so as to obtain at least one fluctuation value of serum creatinine concentration or at least one fluctuation value of eGFR. A first analyzing step is performed by the processor, wherein the at least one fluctuation value of serum creatinine concentration or the at least one fluctuation value of eGFR is analyzed by an AKI assessing model of the processor so as to define the subject as a patient with AKI or a patient without AKI.

According to another aspect of the present disclosure, an acute kidney injury (AKI) assessment system includes a memory and a processor. The memory is for storing a kidney function diagnostic dataset of a subject, wherein the kidney function diagnostic dataset includes at least one serum creatinine concentration data or at least one estimated glomerular filtration rate (eGFR) data, and the at least one serum creatinine concentration data and the at least one estimated glomerular filtration rate data are respectively recorded on 0 to 180 days before an acute kidney injury assessing date. The processor is signally connected to the memory, and the processor includes a preprocessing model and a first analyzing model. The preprocessing model is for calculating a changing degree over a time period of the at least one serum creatinine concentration data or a changing degree over a time period of the at least one estimated glomerular filtration rate data so as to obtain at least one fluctuation value of serum creatinine concentration or at least one fluctuation value of eGFR. The first analyzing model is signally connected to the preprocessing model, wherein the first analyzing model includes an AKI assessing model, and the at least one fluctuation value of serum creatinine concentration or the at least one fluctuation value of eGFR is analyzed by the AKI assessing model so as to define the subject as a patient with AKI or a patient without AKI.

### BRIEF DESCRIPTION OF THE DRAWINGS

The present disclosure can be more fully understood by reading the following detailed description of the embodiment, with reference made to the accompanying drawings as follows:

FIG. 1 is a flow chart of a method for assessing acute kidney injury according to the first embodiment of the present disclosure.

FIG. 2 is a flow chart of a method for assessing acute kidney injury according to the second embodiment of the present disclosure.

FIG. 3 is a flow chart of a method for assessing acute kidney injury according to the third embodiment of the present disclosure.

FIG. 4 is a flow chart of a method for assessing acute kidney injury according to the fourth embodiment of the present disclosure.

FIG. 5 is a flow chart of a method for assessing acute kidney injury according to the fifth embodiment of the present disclosure.

FIG. 6 is a block diagram of an acute kidney injury assessment system according to the sixth embodiment of the present disclosure.

FIG. 7 is a block diagram of an acute kidney injury assessment system according to the seventh embodiment of the present disclosure.

FIG. 8 is a block diagram of an acute kidney injury assessment system according to the eighth embodiment of the present disclosure.

FIG. 9 is a block diagram of an acute kidney injury assessment system according to the ninth embodiment of the present disclosure.

FIG. 10 is a block diagram of an acute kidney injury assessment system according to the tenth embodiment of the present disclosure.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

### <The Method for Assessing Acute Kidney Injury of the Present Disclosure>

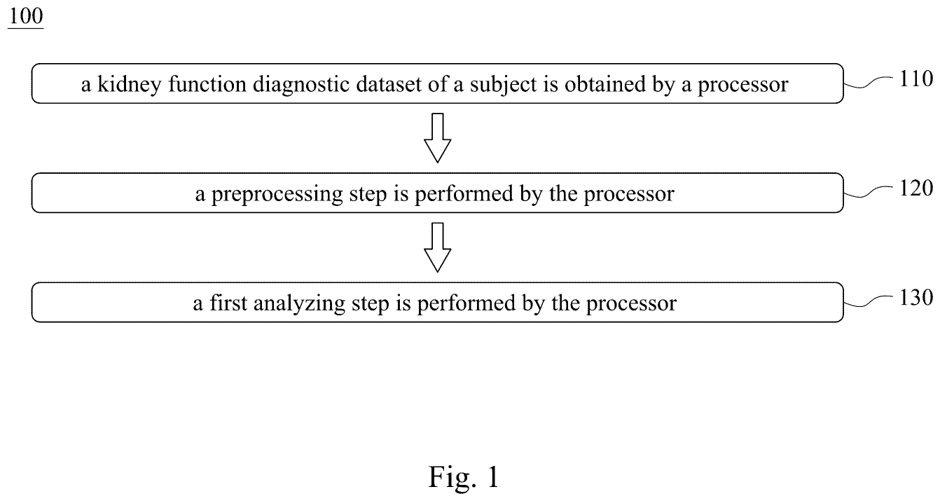

Reference is made to FIG. 1, which is a flow chart of a method 100 for assessing acute kidney injury (AKI) according to the first embodiment of the present disclosure. The method 100 for assessing acute kidney injury includes Step 110, Step 120 and Step 130.

In Step 110, a kidney function diagnostic dataset of a subject is obtained by a processor, wherein the kidney function diagnostic dataset includes at least one serum creatinine concentration data or at least one estimated glomerular filtration rate (eGFR) data, and a recording date of the at least one serum creatinine concentration data and a recording date of the at least one estimated glomerular filtration rate data are respectively on 0 to 180 days before an acute kidney injury assessing date of the subject. In detail, the acute kidney injury assessing date can be the date when the subject goes to the hospital or clinic for outpatient or emergency treatment, or the date in which the renal function of the subject is assessed, the kidney function diagnostic dataset includes at least one of the serum creatinine concentration data recorded on 0 to 180 days before the acute kidney injury assessing date, and the at least one of estimated glomerular filtration rate data can be obtained based on the serum creatinine concentration data corresponding thereto, the age, the sex and the race of the subject according to the Diet in Renal Disease (MDRD) study equation (estimated glomerular filtration rate (eGFR)=186×(Serum creatinine level)^{-1.154}×(age)^{-0.203}×1.212 [if the subject is female, the result must be further×0.742]). The selection of time range within 0 to 180 days before the acute kidney injury assessing date is based on the analysis of current clinical data, and the aforementioned time range represents the longest possible action time that the kidney of older subjects or subjects with chronic kidney disease may be damaged after the administration of nephrotoxic drugs. In other words, the serum creatinine concentration data and the estimated glomerular filtration rate data obtained within the aforementioned time period may be affected by the nephrotoxic drugs, and the subject may be suffered from AKI. Further, the estimated glomerular filtration rate data also can be calculated by other equation except the MDRD study equation, and the present disclosure is not limited thereto. Furthermore, the kidney function diagnostic dataset can be stored in a memory, wherein the memory can be a physical storage device or a cloud storage device, but the present disclosure is not limited thereto. Moreover, the at least one serum creatinine concentration data or at least one estimated glomerular filtration rate data of the kidney function diagnostic dataset can be the data obtained from different medical institutions or the cloud medical records, but the present disclosure is not limited thereto.

In Step 120, a preprocessing step is performed by the processor, wherein a changing degree over a time period of the at least one serum creatinine concentration data or a changing degree over a time period of the at least one estimated glomerular filtration rate data is calculated by the processor so as to obtain at least one fluctuation value of serum creatinine concentration or at least one fluctuation value of eGFR.

In Step 130, a first analyzing step is performed by the processor, wherein the at least one fluctuation value of serum creatinine concentration or the at least one fluctuation value of eGFR is analyzed by the AKI assessing model so as to define the subject as a patient with AKI and a patient without AKI. Further, the AKI assessing model can be established by a Cox regression calculating classifier.

Therefore, by analyzing the fluctuation value of the serum creatinine concentration data or the fluctuation value of the estimated glomerular filtration rate data recorded on 0 to 180 days before the acute kidney injury assessing date by the AKI assessing model, the method 100 for assessing acute kidney injury of the present disclosure can effectively and accurately assess the AKI status of the subject. Thus, not only is it favorable for formulating the design of subsequent medical plans, but also the long-term impairment of kidney function caused by delays in treatment can be avoided. Hence, the method 100 for assessing acute kidney injury has excellent clinical application potential.

Further, in the method 100 for assessing acute kidney injury of the present disclosure, the patient with AKI has the at least one fluctuation value of serum creatinine concentration larger than or equal to 50% or has the at least one fluctuation value of eGFR larger than or equal to 35%. In detail, the patient with AKI has a greater degree of deterioration in the renal function within 0 to 180 days before the acute kidney injury assessing date, and when the subject is defined as the patient with AKI by the AKI assessing model of the present disclosure, the fluctuation value of serum creatinine concentration can be larger than or equal to 50% or the fluctuation value of eGFR can be larger than or equal to 35%. At this time, the patient with AKI predicted by the AKI assessing model of the present disclosure will be referred to the nephrologist to treat timely. On the contrary, when the fluctuation value of serum creatinine concentration of the subject is smaller than 50% or the fluctuation value of eGFR is smaller than 35%, the change of the renal function of the subject on 0 to 180 days before the acute kidney injury assessing date is still within the standard value range, and the subject is defined as the patient without AKI.

Further, in the method 100 for assessing acute kidney injury of the present disclosure, the at least one serum creatinine concentration data can include a maximum serum creatinine concentration data and a minimum serum creatinine concentration data, and the at least one estimated glomerular filtration rate data can include a maximum estimated glomerular filtration rate data and a minimum estimated glomerular filtration rate data. The fluctuation value of serum creatinine concentration is calculated based on the maximum serum creatinine concentration data and the minimum serum creatinine concentration data according to the fluctuation value of serum creatinine concentration calculating formula (I), and the fluctuation value of eGFR is calculated based on the maximum estimated glomerular filtration rate data and the minimum estimated glomerular filtration rate data according to the fluctuation value of eGFR calculating formula (II).

The fluctuation value of serum creatinine concentration calculating formula (I) is shown as follows:

$$( Formula I )$$

$$Fluctuation value of serum creatinine concentration ( \% ) = ( \frac{\vert {SCr}_{max} - {SCr}_{min} \vert}{{SCr}_{min}} ) \times 100 \% ,$$

wherein SCrₘₐₓ represents the value of the maximum serum creatinine concentration data, and SCrₘᵢₙ represents the value of the minimum serum creatinine concentration data.

The fluctuation value of eGFR calculating formula (II) is shown as follows:

$$( Formula II )$$

$$Fluctuation value of eGFR ( \text{?} = \text{?} \frac{\vert {eGFR}_{max} - {eGFR}_{min} \vert}{{eGFR}_{max}} ) \times 100 \% ;$$

$$\text{?} \text{indicates text missing or illegible when filed}$$

wherein eGFRₘₐₓ represents the value of the maximum estimated glomerular filtration rate data, and the eGFRₘᵢₙ represents the value of the minimum estimated glomerular filtration rate data.

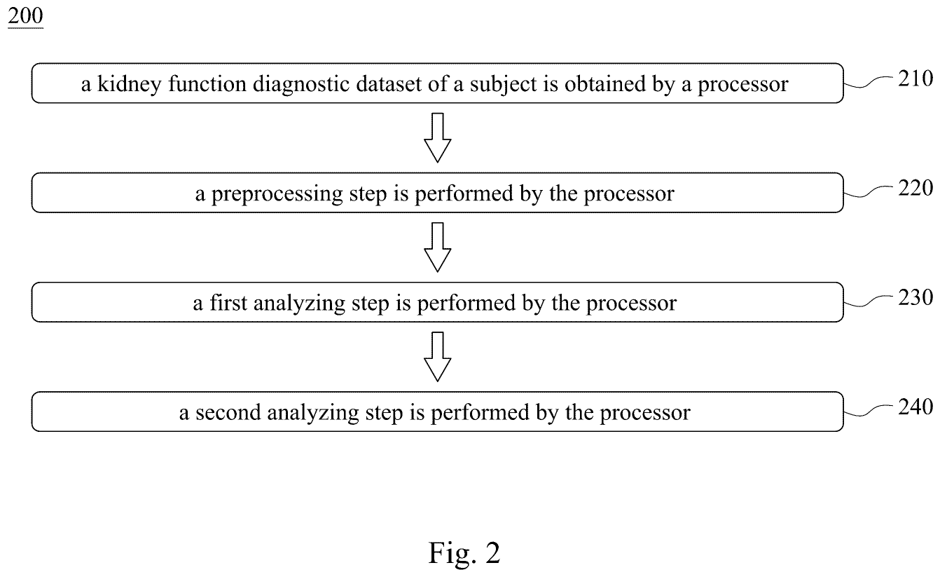

Reference is made to FIG. 2, which is a flow chart of a method 200 for assessing acute kidney injury according to the second embodiment of the present disclosure. The method 200 for assessing acute kidney injury includes Step 210, Step 220, Step 230 and Step 240, wherein Step 210, Step 220 and Step 230 are the same as Step 110, Step 120 and Step 130 of the method 100 for assessing acute kidney injury of FIG. 1, so that the same details there between are not described again herein.

In Step 240, a second analyzing step is performed by the processor. In detail, the kidney function diagnostic dataset of the present disclosure can further include a base serum creatinine concentration data, and the at least one serum creatinine concentration data can include a first serum creatinine concentration data, wherein a recording date of the base serum creatinine concentration data is the acute kidney injury assessing date, and the recording date of the first serum creatinine concentration data is the closest to the acute kidney injury assessing date in the at least one serum creatinine concentration data.

In the second analyzing step, the base serum creatinine concentration data and the first serum creatinine concentration data are calculated by the processor so as to obtain a difference value of serum creatinine concentration, and the difference value of serum creatinine concentration is analyzed by the AKI assessing model so as to assess that the patient with AKI is a deteriorating AKI patient or a stable AKI patient. In detail, the first serum creatinine concentration data can be the closest serum creatinine concentration data (that is, the last trackbacking serum creatinine concentration data) obtained before the acute kidney injury assessing date, the serum creatinine concentration data obtained on the acute kidney injury assessing date is the base serum creatinine concentration data, and the recording date of the base serum creatinine concentration data must be later than the recording date of the first serum creatinine concentration data. That is, the second analyzing step can further analyze the difference of two of the serum creatinine concentration data recorded before the acute kidney injury assessing date by the processor so as to obtain the type of the patient with AKI.

Further, in the method 200 for assessing acute kidney injury of the present disclosure, if the base serum creatinine concentration data cannot be obtained in time, the first and the second serum creatinine concentration data that are recorded before the acute kidney injury assessing date and are the closest to the acute kidney injury assessing date will be captured, and the difference there between will be calculated by the processor so as to obtain a difference value of serum creatinine concentration.

Further, in the method 200 for assessing acute kidney injury of the present disclosure, the deteriorating AKI patient has the difference value of serum creatinine concentration larger than or equal to 0.3 mg/dl, and the stable AKI patient has the difference value of serum creatinine concentration smaller than 0.3 mg/dl. In detail, the difference value of serum creatinine concentration is calculated according to the difference value of serum creatinine concentration calculating formula (III), and the difference value of serum creatinine concentration calculating formula (III) is shown as follows:

$$( Formula III )$$

$$Difference value of serum creatinine concentration ( \% ) = {SCr}_{Last} - {SCr}_{Last - 1} ,$$

wherein SCr_{Last} represents the value of the base serum creatinine concentration data, and SCr_{Last-1} represents the value of the first serum creatinine concentration data. If the difference value of serum creatinine concentration is larger than or equal to 0.3 mg/dl, it represents that the serum creatinine concentration of the patient with AKI has a larger increase and deterioration before the acute kidney injury assessing date, and the medical status of the patient with AKI is rapidly deteriorating. Accordingly, the patient with AKI is defined as a deteriorating AKI patient by the AKI assessing model of the present disclosure. On the contrary, if the difference value of serum creatinine concentration is smaller than 0.3 mg/dl, the medical status of the patient with AKI is without rapidly deteriorating, so that the patient with AKI is defined as a stable AKI patient by the AKI assessing model of the present disclosure.

Therefore, by analyzing the fluctuation values of the serum creatinine concentration data and the estimated glomerular filtration rate data recorded on 0 to 180 days before the acute kidney injury assessing date by the AKI assessing model, the method 200 for assessing acute kidney injury of the present disclosure can assess the AKI status of the subject so as to facilitate the design of subsequent medical plans. Hence, the method 200 for assessing acute kidney injury has excellent clinical application potential.

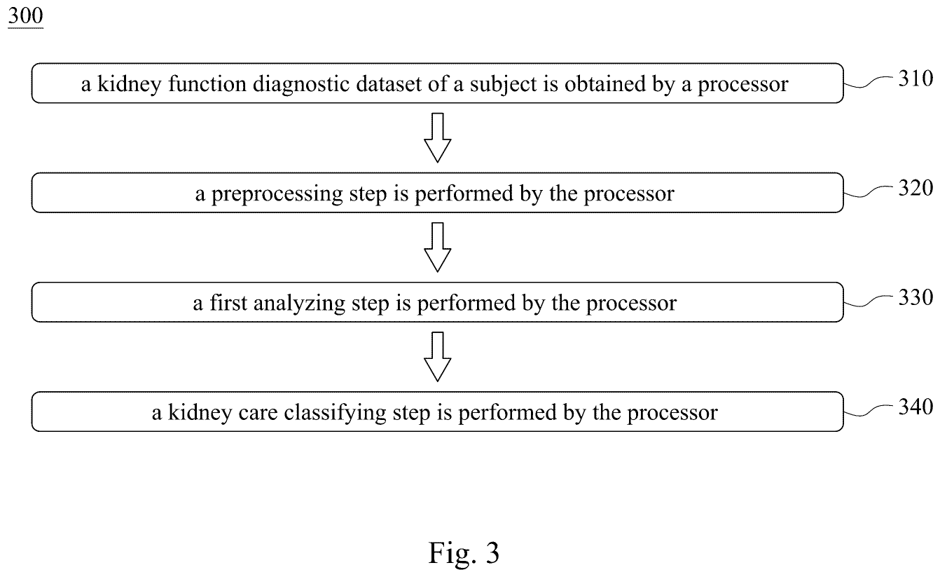

Reference is made to FIG. 3, which is a flow chart of a method 300 for assessing acute kidney injury according to the third embodiment of the present disclosure. The method 300 for assessing acute kidney injury includes Step 310, Step 320, Step 330 and Step 340, wherein Step 310, Step 320 and Step 330 are the same as Step 110, Step 120 and Step 130 of the method 100 for assessing acute kidney injury of FIG. 1, so that the same details there between are not described again herein.

In Step 340, a kidney care classifying step is performed by the processor. In detail, the kidney function diagnostic dataset can further include a base estimated glomerular filtration rate data, and a recording date of the base estimated glomerular filtration rate data is the acute kidney injury assessing date. In the kidney care classifying step, the base estimated glomerular filtration rate data is analyzed by the AKI assessing model of the present disclosure so as to obtain a result of kidney care classification. In detail, the kidney care classifying step can be used to respectively analyze the values of the base estimated glomerular filtration rate data of the patient without AKI, the deteriorating AKI patient and the stable AKI patient and then output the corresponding results of kidney care classification. Further, the doctors of outpatient clinics and emergency department can design appropriate medical plans according to the aforementioned results of kidney care classification so as to prevent the long-term damage to the renal function caused by delays in treatment.

Please refer to Table 1, which shows the values of base estimated glomerular filtration rate data and the corresponding results of kidney care classification thereof.

TABLE 1

| | Risk of dialysis within 3 years | | |
|---|---|---|---|
| Base eGFR | Patient | Stable AKI | Deteriorating |
| (ml/min/1.73 m²) | without AKI | patient | AKI patient |
| eGFR < 45 | 20 times < | 20 times < | 50 times < |
| | Referral to | Referral to | Mandatory referral |
| | nephrology | nephrology | to nephrology |
| 45 ≤ eGFR < 60 | <5 times | 5~10 times | 5~10 times |
| | Track renal | Track renal | Track renal |
| | function | function or | function or |
| | | referral to | referral to |
| | | nephrology | nephrology |
| 60 ≤ eGFR | Normal renal | <5 times | <5 times |
| | function | Track renal | Track renal |
| | | function | function |

As shown in Table 1, when the subject is a patient with AKI (including the stable AKI patient and the deteriorating AKI patient), the patient with AKI will be referred to the nephrologist when the base estimated glomerular filtration rate data thereof is smaller than 60 ml/min/1.73 m², and if the base estimated glomerular filtration rate data of the deteriorating AKI patient is smaller than 45 ml/min/1.73 m², the deteriorating AKI patient will be mandatory referred to the nephrologist. Further, the patient without AKI will also be referred to the nephrologist when the base estimated glomerular filtration rate data thereof is smaller than 60 ml/min/1.73 m². Therefore, by analyzing the last estimated glomerular filtration rate data recorded before the acute kidney injury assessing date or analyzing the base estimated glomerular filtration rate data recorded on the acute kidney injury assessing date by the AKI assessing model, the method 300 for assessing acute kidney injury can output a corresponding result of kidney care classification. Hence, it is favorable for designing the subsequent medical plans by the doctor, and thus the method 300 for assessing acute kidney injury has excellent clinical application potential.

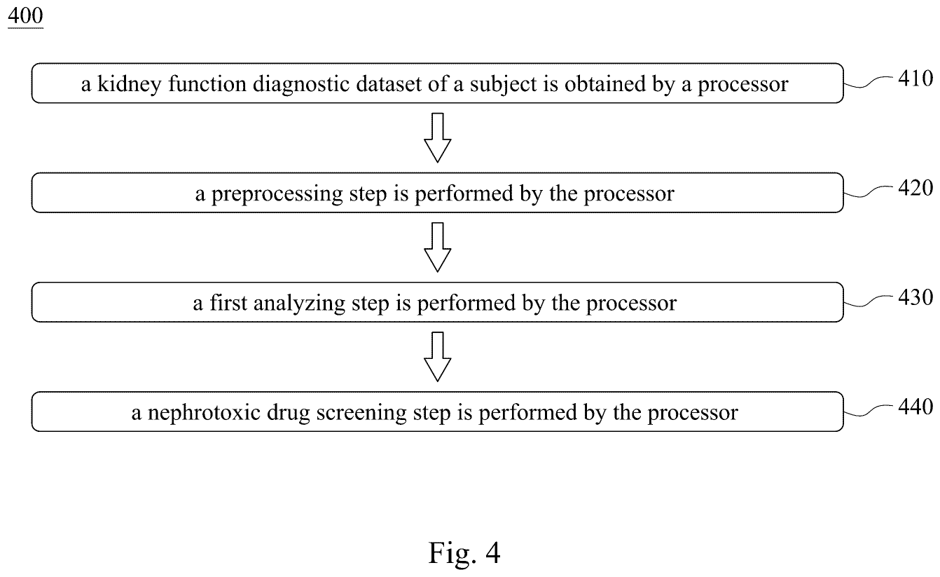

Reference is made to FIG. 4, which is a flow chart of a method 400 for assessing acute kidney injury according to the fourth embodiment of the present disclosure. The method 400 for assessing acute kidney injury includes Step 410, Step 420, Step 430 and Step 440, wherein Step 410, Step 420 and Step 430 are the same as Step 110, Step 120 and Step 130 of the method 100 for assessing acute kidney injury of FIG. 1, so that the same details there between are not described again herein.

In Step 440, a nephrotoxic drug screening step is performed by the processor, wherein the kidney function diagnostic dataset can further include a drug utilization data, and the drug utilization data is analyzed by the AKI assessing model in the nephrotoxic drug screening step so as to output a screening result of nephrotoxic drug usage. In detail, the nephrotoxic drug includes six kinds of drugs including non-steroidal anti-inflammatory drugs (NSAIDs), radiocontrast, antimicrobials, chemotherapy and immunotherapy, renin-angiotension system blocker (ARB/ACEi) and diuretics which can affect the renal function of a subject, so that it is favorable for analyzing and assessing whether the cause of AKI of the subject is due to the influences of nephrotoxic drugs. Further, if the subject has suffered from the acute kidney injury on 0 to 180 days before the acute kidney injury assessing date, the screening result of nephrotoxic drug usage also can be provided as a warning for physicians to prescribe medication. Accordingly, the method 400 for assessing acute kidney injury of the present disclosure has excellent clinical application potential.

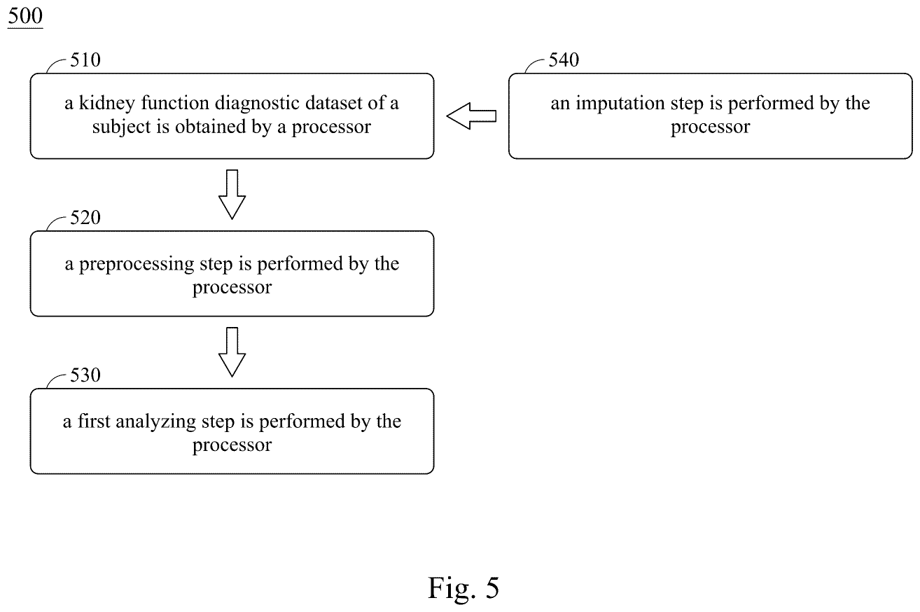

Reference is made to FIG. 5, which is a flow chart of a method 500 for assessing acute kidney injury according to the fifth embodiment of the present disclosure. The method 500 for assessing acute kidney injury includes Step 510, Step 520, Step 530 and Step 540, wherein Step 510, Step 520 and Step 530 are the same as Step 110, Step 120 and Step 130 of the method 100 for assessing acute kidney injury of FIG. 1, so that the same details there between are not described again herein.

In Step 540, an imputation step is performed by the processor, wherein the kidney function diagnostic dataset further includes a physiological data and a biomedical data of the subject, and the physiological data and the biomedical data of the subject are analyzed by an imputation model of the processor so as to obtain the at least one serum creatinine concentration data or the at least one estimated glomerular filtration rate data. In detail, the imputation step is for the subject who is without the serum creatinine concentration data or the estimated glomerular filtration rate in the kidney function diagnostic dataset. At this time, the imputation model will analyze the physiological data and the biomedical data of the subject to perform an imputation process to obtain the serum creatinine concentration data or the estimated glomerular filtration rate.

Further, the imputation model of the present disclosure can be established by an XGBoost regression classifier, and the physiological data and the biomedical data are respectively processed with a Bayesian optimization process by the imputation model so as to obtain a feature set of the imputation model. In detail, the Bayesian optimization process is a sequential design strategy for global optimization of black-box functions, and it does not assume any functional forms. It is usually employed to optimize expensive-to-evaluate functions. Thus, by analyzing the physiological data and the biomedical data with the Bayesian optimization process, an optimal model configuration of the imputation model can be obtained, and the hyper-parameter tuning through multiple rounds of training can be achieved by the combination of the Bayesian optimization process and the XGBoost regression classifier so as to obtain the imputation model with high accuracy.

Furthermore, in the method 500 for assessing acute kidney injury of the present disclosure, the physiological data can include the information of the age and the sex of the subject, the biomedical data can include a value of blood urine nitrogen (BUN), a value of serum phosphorus (P), a value of serum iron, a value of urine protein, a value of urine albumin-to-creatinine ratio (uACR), a value of hemoglobin, a value of serum uric acid, a value of serum potassium, a value of serum albumin, a value of serum calcium, a value of serum sodium, a value of serum triglyceride (TG) and a value of serum cholesterol, etc., and the composition of the feature set of the imputation model can be further adjusted by the Bayesian optimization process, so that the result output by the imputation model of the present disclosure can be more in line with the actual status of the subject so as to enhance the assessing accuracy of the method 500 for assessing acute kidney injury of the present disclosure.

### <The Acute Kidney Injury Assessment System of the Present Disclosure>

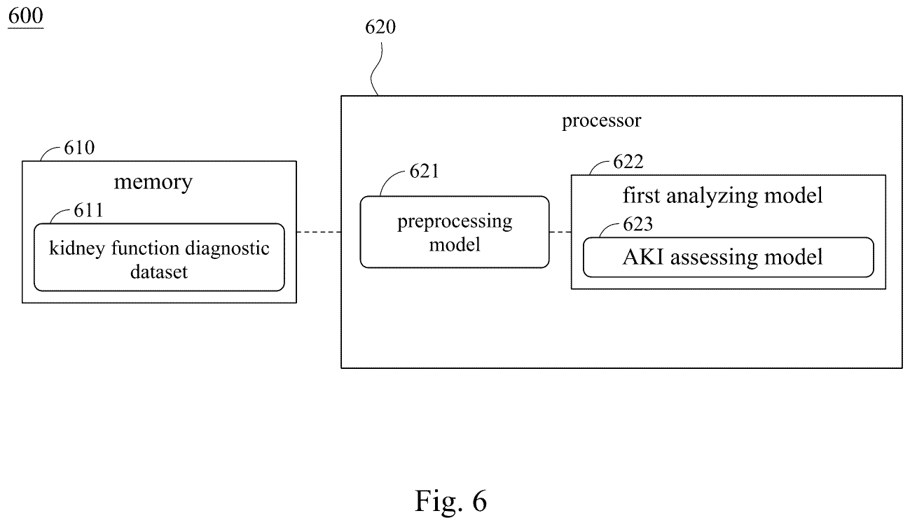

Reference is made to FIG. 6, which is a block diagram of an acute kidney injury assessment system 600 according to the sixth embodiment of the present disclosure. The acute kidney injury assessment system 600 includes a memory 610 and a processor 620.

The memory 610 is for storing a kidney function diagnostic dataset 611 of a subject, wherein the kidney function diagnostic dataset 611 includes at least one serum creatinine concentration data or at least one estimated glomerular filtration rate data, and the at least one serum creatinine concentration data and the at least one estimated glomerular filtration rate data are respectively recorded on 0 to 180 days before an acute kidney injury assessing date. In detail, the acute kidney injury assessing date can be the date when the subject goes to the hospital or clinic for outpatient or emergency treatment, or the date in which the renal function of the subject will be assessed for the following assessment. Furthermore, the kidney function diagnostic dataset 611 includes at least one of the serum creatinine concentration data recorded on 0 to 180 days before the acute kidney injury assessing date, and the at least one of estimated glomerular filtration rate data can be obtained based on the serum creatinine concentration data corresponding thereto by the MDRD study equation. According to the analysis of current clinical data, the time range that the kidney of older subjects or subjects with chronic kidney disease may be damaged is 0 to 180 days after the potential exposure to nephrotoxic drugs, and the serum creatinine concentration data and the estimated glomerular filtration rate data obtained within the aforementioned time period may be affected by the nephrotoxic drugs, so that the subject may be suffered from AKI and can be used to analyze the AKI status of the subject. Furthermore, the kidney function diagnostic dataset 611 can be stored in a physical storage device or a cloud storage device, but the present disclosure is not limited thereto. Moreover, the at least one serum creatinine concentration data or at least one estimated glomerular filtration rate data of the kidney function diagnostic dataset can be the data obtained from different medical institutions or the cloud medical record, but the present disclosure is not limited thereto.

The processor 620 is signally connected to the memory 610, wherein the processor 620 includes a preprocessing model 621 and a first analyzing model 622.

The preprocessing model 621 is for calculating a changing degree over a time period of the at least one serum creatinine concentration data or a changing degree over a time period of the at least one estimated glomerular filtration rate data so as to obtain at least one fluctuation value of serum creatinine concentration or at least one fluctuation value of eGFR.

The first analyzing model 622 is signally connected to the preprocessing model 621, wherein the first analyzing model 622 includes an AKI assessing model 623, and the at least one fluctuation value of serum creatinine concentration or the at least one fluctuation value of eGFR is analyzed by the AKI assessing model 623 so as to define the subject as a patient with AKI or a patient without AKI. Further, the AKI assessing model 623 can be established by a Cox regression calculating classifier.

In the acute kidney injury assessment system 600 of the present disclosure, the patient with AKI has the at least one fluctuation value of serum creatinine concentration larger than or equal to 50% or has the at least one fluctuation value of eGFR larger than or equal to 35%. In detail, the patient with AKI has a greater degree of deterioration in the renal function within 0 to 180 days before the acute kidney injury assessing date, and when the subject is defined as the patient with AKI by the AKI assessing model 623 of the present disclosure, the fluctuation value of serum creatinine concentration can be larger than or equal to 50% or the fluctuation value of eGFR can be larger than or equal to 35%. At this time, the patient with AKI predicted by the AKI assessing model 623 of the present disclosure will be referred to the nephrologist to treat timely. On the contrary, when the fluctuation value of serum creatinine concentration of the subject is smaller than 50% or the fluctuation value of eGFR is smaller than 35%, the change of the renal function of the subject on 0 to 180 days before the acute kidney injury assessing date is still within the standard value range, and the subject is defined as the patient without AKI.

Further, in the acute kidney injury assessment system 600 of the present disclosure, the at least one serum creatinine concentration data can include a maximum serum creatinine concentration data and a minimum serum creatinine concentration data, and the at least one estimated glomerular filtration rate data can include a maximum estimated glomerular filtration rate data and a minimum estimated glomerular filtration rate data. The fluctuation value of serum creatinine concentration is calculated based on the maximum serum creatinine concentration data and the minimum serum creatinine concentration data according to the aforementioned fluctuation value of serum creatinine concentration calculating formula (I), and the fluctuation value of eGFR is calculated based on the maximum estimated glomerular filtration rate data and the minimum estimated glomerular filtration rate data according to the aforementioned fluctuation value of eGFR calculating formula (II). The details of the fluctuation value of serum creatinine concentration calculating formula (I) and the fluctuation value of eGFR calculating formula (II) are shown in the aforementioned description, and they are not described again herein.

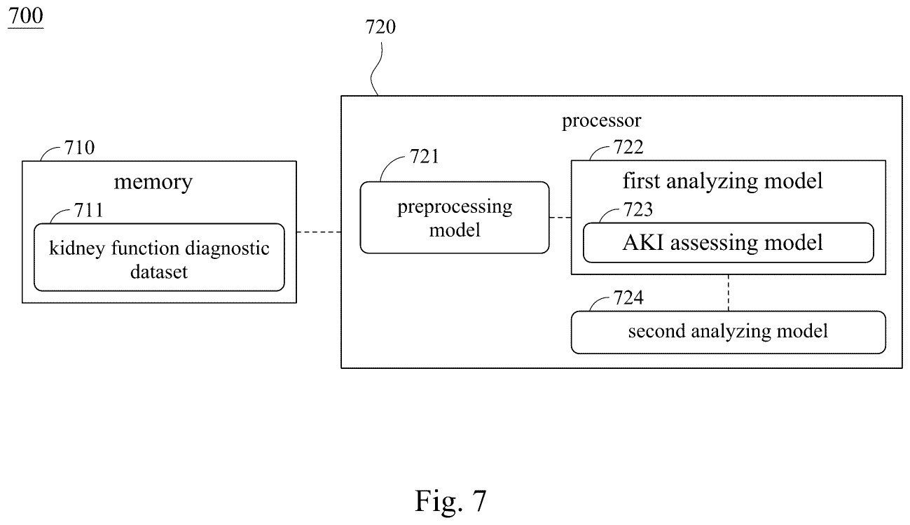

Reference is made to FIG. 7, which is a block diagram of an acute kidney injury assessment system 700 according to the seventh embodiment of the present disclosure. The acute kidney injury assessment system 700 includes a memory 710 and a processor 720. The memory 710 is for storing a kidney function diagnostic dataset 711 of a subject, and the processor 720 is signally connected to the memory 710 and includes a preprocessing model 721, a first analyzing model 722 and a second analyzing model 724, wherein the memory 710 is similar with the memory 610 of the acute kidney injury assessment system 600 of FIG. 6, and the same details there between are not described again herein.

The first analyzing model 722 is signally connected to the preprocessing model 721, and the first analyzing model 722 includes an AKI assessing model 723, wherein the first analyzing model 722 is the same as the first analyzing model 622 of the acute kidney injury assessment system 600 of FIG. 6, and the same details there between are not described again herein. In detail, the kidney function diagnostic dataset 711 can further include a base serum creatinine concentration data, and the at least one serum creatinine concentration data can include a first serum creatinine concentration data, wherein a recording date of the base serum creatinine concentration data is the acute kidney injury assessing date, and the recording date of the first serum creatinine concentration data is the closest to the acute kidney injury assessing date in the at least one serum creatinine concentration data.

The second analyzing model 724 is signally connected to the first analyzing model 722, wherein the base serum creatinine concentration data and the first serum creatinine concentration data are calculated by the second analyzing model 624 so as to obtain a difference value of serum creatinine concentration, and the difference value of serum creatinine concentration is analyzed by the AKI assessing model 723 so as to assess that the patient with AKI is a deteriorating AKI patient or a stable AKI patient. In detail, the recording date of the base serum creatinine concentration data is the acute kidney injury assessing date, and the recording date of the first serum creatinine concentration data is the closest to the acute kidney injury assessing date among all of the serum creatinine concentration data. The first serum creatinine concentration data can be the closest serum creatinine concentration data obtained before the acute kidney injury assessing date, the serum creatinine concentration data obtained on the acute kidney injury assessing date is the base serum creatinine concentration data, and the recording date of the base serum creatinine concentration data must be later than the recording date of the first serum creatinine concentration data. That is, the second analyzing model 724 can further analyze the difference of two of the serum creatinine concentration data recorded before the acute kidney injury assessing date, and the AKI assessing model 723 can further analyze the difference value of serum creatinine concentration so as to obtain the type of the patient with AKI.

Further, in the acute kidney injury assessment system 700 of the present disclosure, if the base serum creatinine concentration data cannot be obtained in time, the first and the second serum creatinine concentration data that are recorded before the acute kidney injury assessing date and are the closest to the acute kidney injury assessing date will be captured, and the difference there between will be calculated by the processor so as to obtain a difference value of serum creatinine concentration. Furthermore, the difference value of serum creatinine concentration is calculated according to the difference value of serum creatinine concentration calculating formula (III). The details of the difference value of serum creatinine concentration calculating formula (III) are shown in the aforementioned description, and it is not described again herein.

Further, in the acute kidney injury assessment system 700, the deteriorating AKI patient has the difference value of serum creatinine concentration larger than or equal to 0.3 mg/dL, and the stable AKI patient has the difference value of serum creatinine concentration smaller than 0.3 mg/dl. If the difference value of serum creatinine concentration is larger than or equal to 0.3 mg/dL, it represents that the serum creatinine concentration of the patient with AKI has a larger increase and deterioration before the acute kidney injury assessing date, and the medical status of the patient with AKI is rapidly deteriorating. Accordingly, the patient with AKI is defined as a deteriorating AKI patient by the AKI assessing model 723 of the present disclosure. On the contrary, if the difference value of serum creatinine concentration is smaller than 0.3 mg/dl, the medical status of the patient with AKI is without rapidly deteriorating, so that the patient with AKI is defined as a stable AKI patient by the AKI assessing model 723 of the present disclosure.

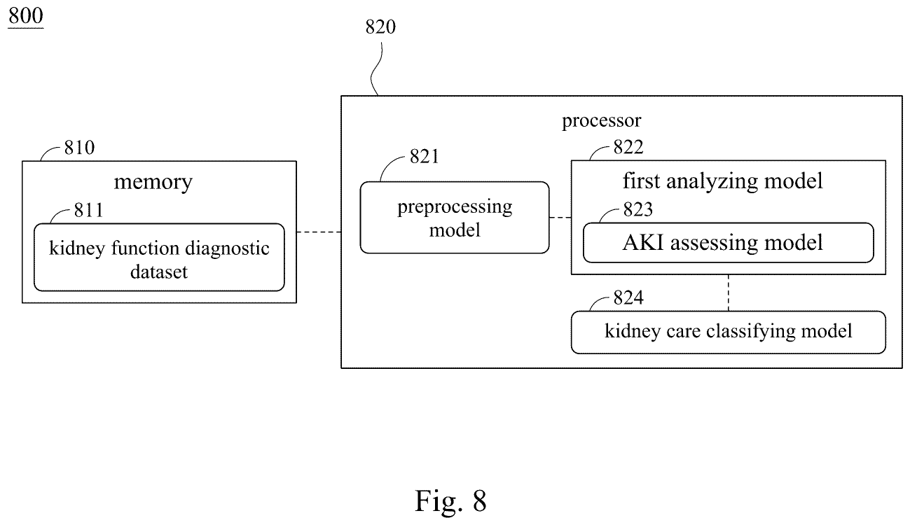

Reference is made to FIG. 8, which is a block diagram of an acute kidney injury assessment system 800 according to the eighth embodiment of the present disclosure. The acute kidney injury assessment system 800 includes a memory 810 and a processor 820. The memory 810 is for storing a kidney function diagnostic dataset 811 of a subject, and the processor 820 is signally connected to the memory 810 and includes a preprocessing model 821, a first analyzing model 822 and a kidney care classifying model 824, wherein the memory 810 is similar with the memory 610 of the acute kidney injury assessment system 600 of FIG. 6, and the same details there between are not described again herein.

The first analyzing model 822 is signally connected to the preprocessing model 821, and the first analyzing model 822 includes an AKI assessing model 823. In detail, the kidney function diagnostic dataset 811 can further include a base estimated glomerular filtration rate data, and a recording date of the base estimated glomerular filtration rate data is the acute kidney injury assessing date. The kidney care classifying model 824 is for analyzing the base estimated glomerular filtration rate data by the AKI assessing model 823 of the present disclosure so as to obtain a result of kidney care classification.

In detail, the base estimated glomerular filtration rate data is recorded the closest to the acute kidney injury assessing date or is recorded on the acute kidney injury assessing date, and the AKI assessing model 823 of the present disclosure can respectively analyze the values of the base estimated glomerular filtration rate data of the patient without AKI, the deteriorating AKI patient and the stable AKI patient and then output the corresponding results of kidney care classification. Further, the doctors of outpatient clinics and emergency department can design appropriate medical plans according to the aforementioned results of kidney care classification so as to prevent the long-term damage to the renal function caused by delays in treatment. Moreover, the values of base estimated glomerular filtration rate data and the corresponding results of kidney care classification thereof as shown in Table 1, and they are not described again herein.

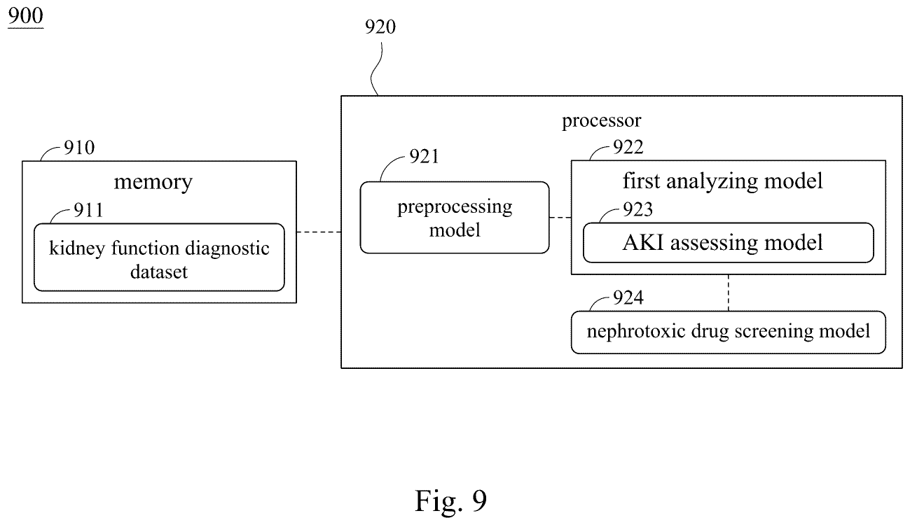

Reference is made to FIG. 9, which is a block diagram of an acute kidney injury assessment system 900 according to the ninth embodiment of the present disclosure. The acute kidney injury assessment system 900 includes a memory 910 and a processor 920. The memory 910 is for storing a kidney function diagnostic dataset 911 of a subject, and the processor 920 is signally connected to the memory 910 and includes a preprocessing model 921, a first analyzing model 922 and a nephrotoxic drug screening model 924, wherein the memory 910 is similar with the memory 610 of the acute kidney injury assessment system 600 of FIG. 6, and the same details there between are not described again herein.

The first analyzing model 922 is signally connected to the preprocessing model 921, and the first analyzing model 922 includes an AKI assessing model 923, wherein the first analyzing model 922 is the same as the first analyzing model 622 of the acute kidney injury assessment system 600 of FIG. 6, and the same details there between are not described again herein. In detail, the kidney function diagnostic dataset 911 can further include a drug utilization data, and the nephrotoxic drug screening model 924 is for analyzing the drug utilization data so as to output a screening result of nephrotoxic drug usage. Thus, it is favorable for analyzing and assessing whether the cause of AKI of the subject is due to the influences of non-steroidal anti-inflammatory drugs, radiocontrast, antimicrobials, chemotherapy and immunotherapy, renin-angiotension system blocker, diuretics and other nephrotoxic drugs, so that the acute kidney injury assessment system 900 of the present disclosure has excellent clinical application potential. Further, if the subject has suffered from the acute kidney injury on 0 to 180 days before the acute kidney injury assessing date, the screening result of nephrotoxic drug usage also can be provided as a warning for physicians to prescribe medication.

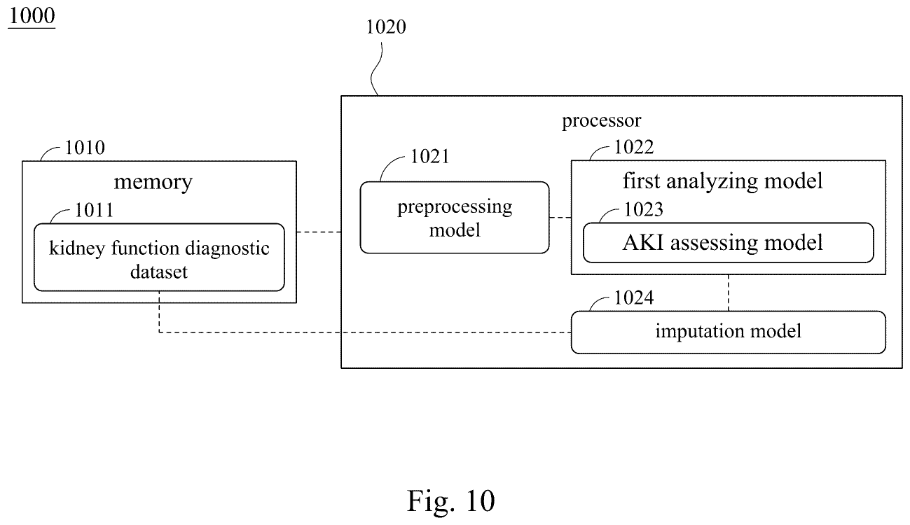

Reference is made to FIG. 10, which is a block diagram of an acute kidney injury assessment system 1000 according to the tenth embodiment of the present disclosure. The acute kidney injury assessment system 1000 includes a memory 1010 and a processor 1020. The memory 1010 is for storing a kidney function diagnostic dataset 1011 of a subject, and the processor 1020 is signally connected to the memory 1010 and includes a preprocessing model 1021, a first analyzing model 1022 and an imputation model 1024, wherein the memory 1010 is similar with the memory 610 of the acute kidney injury assessment system 600 of FIG. 6, and the same details there between are not described again herein.

The first analyzing model 1022 is signally connected to the preprocessing model 1021, and the first analyzing model 1022 includes an AKI assessing model 1023, wherein the first analyzing model 1022 is the same as the first analyzing model 622 of the acute kidney injury assessment system 600 of FIG. 6, and the same details there between are not described again herein. In detail, in the acute kidney injury assessment system 1000 of the present disclosure, the kidney function diagnostic dataset 1011 can further include a physiological data and a biomedical data of the subject, and the physiological data and the biomedical data of the subject are analyzed by the imputation model 1024 of the processor so as to obtain the at least one serum creatinine concentration data or the at least one estimated glomerular filtration rate data. In detail, the imputation model 1024 is for the subject who is without the serum creatinine concentration data or the estimated glomerular filtration rate in the kidney function diagnostic dataset. At this time, the imputation model 1024 will analyze the physiological data and the biomedical data of the subject to perform an imputation process to obtain the serum creatinine concentration data or the estimated glomerular filtration rate.

Further, the imputation model 1024 of the present disclosure can be established by an XGBoost regression classifier, and the physiological data and the biomedical data are respectively processed with a Bayesian optimization process by the imputation model 1024 so as to obtain a feature set of the imputation model 1024. In detail, the Bayesian optimization process is a sequential design strategy for global optimization of black-box functions, and it does not assume any functional forms. It is usually employed to optimize expensive-to-evaluate functions. Thus, by analyzing the physiological data and the biomedical data with the Bayesian optimization process, an optimal model configuration of the imputation model 1024 can be obtained, and the hyper-parameter tuning through multiple rounds of training can be achieved by the combination of the Bayesian optimization process and the XGBoost regression classifier so as to obtain the imputation model 1024 with high accuracy.

Furthermore, in the acute kidney injury assessment system 1000 of the present disclosure, the physiological data can include the information of the age and the sex of the subject, the biomedical data can include a value of blood urine nitrogen, a value of serum phosphorus, a value of serum iron, a value of urine protein, a value of urine albumin-to-creatinine ratio, a value of hemoglobin, a value of serum uric acid, a value of serum potassium, a value of serum albumin, a value of serum calcium, a value of serum sodium, a value of serum triglyceride and a value of serum cholesterol, etc., and the composition of the feature set of the imputation model 1024 can be further adjusted by the Bayesian optimization process, so that the result output by the imputation model 1024 of the present disclosure can be more in line with the actual status of the subject so as to enhance the assessing accuracy of the acute kidney injury assessment system 1000 of the present disclosure.

Therefore, by analyzing the fluctuation values of the serum creatinine concentration data and the estimated glomerular filtration rate data recorded on 0 to 180 days before the acute kidney injury assessing date by the AKI assessing model, the acute kidney injury assessment system 600, the acute kidney injury assessment system 700, the acute kidney injury assessment system 800, the acute kidney injury assessment system 900 and the acute kidney injury assessment system 1000 of the present disclosure can assess the acute kidney injury status of the subject. Furthermore, the acute kidney injury assessment system 600, the acute kidney injury assessment system 700, the acute kidney injury assessment system 800, the acute kidney injury assessment system 900 and the acute kidney injury assessment system 1000 of the present disclosure can further calculate the prognostic risk instantly and then obtain the possible cause of AKI of the subject by calculating the risk matrix by the AKI assessing model and then generalizing the conditions of the comprehensive type of AKI, results of kidney care classification and the nephrotoxic drug usage of the patient with AKI. Hence, it is favorable for designing the subsequent medical plans, and the long-term impairment of kidney function caused by delays in treatment can be avoided, so that the acute kidney injury assessment system 600, the acute kidney injury assessment system 700, the acute kidney injury assessment system 800, the acute kidney injury assessment system 900 and the acute kidney injury assessment system 1000 have excellent clinical application potential.

### Example

### I. Kidney Function Diagnostic Dataset

The kidney function diagnostic dataset used in the establishment of the AKI assessing model of the present test is collected by China Medical University Hospital and includes 518,149 patients suffered from the end-stage renal disease (ESRD) in outpatient visits, and the patients are aged from 20 to 90 years old. After excluding the patients aged<18 or >90 years at an appointed outpatient visit, with a history of the end stage kidney disease (“ESKD” hereafter), with a history of cancer, having unreasonable data of death date and having insufficient serum creatinine concentration data to determine the acute kidney disease, there is a total of 94,733 patients used to analyze the efficiency of the AKI assessing model. In the 94,733 patients, 79,423 patients are without AKI, 13,512 patients are stable AKI patients, and 1,798 patients are deteriorating AKI patients.

### II. The Credibility for Assessing the Acute Kidney Injury of the Method for Assessing Acute Kidney Injury and the Acute Kidney Injury Assessment System of the Present Disclosure

In the 94,733 patients, 56,839 patients are served as the training dataset, 18,947 patients are served as the testing dataset, and 18,947 patients are served as the validation dataset. Further, in the present test, the AKI assessing model is established by a Cox regression calculating classifier based on the training dataset, wherein the training features of the Cox regression calculating classifier include the age, the sex, the serum creatinine concentration data and the estimated glomerular filtration rate data of the subject, and the credibility for acute kidney injury of the AKI assessing model is analyzed. Further, the training features of the Cox regression calculating classifier can further include the record of comorbidity and the using history of the contract agent, but the present disclosure is not limited thereto.

Reference is made to Table 2, which shows the verifying results of the occurrence of acute kidney injury obtained by the AKI assessing model of the method for assessing acute kidney injury and the acute kidney injury assessment system of the present disclosure based on the binary classification method.

TABLE 2

| | Training dataset | Testing dataset | Validation dataset |
|---|---|---|---|
| AUC | 0.88 | 0.82 | 0.82 |
| Threshold | 0.166 | 0.171 | 0.145 |
| Accuracy | 0.824 | 0.806 | 0.777 |
| Precision | 0.476 | 0.434 | 0.386 |
| Sensitivity | 0.756 | 0.683 | 0.701 |
| Specificity | 0.837 | 0.83 | 0.791 |
| F-score | 0.584 | 0.53 | 0.498 |
| MCC | 0.5 | 0.43 | 0.4 |
| Kappa | 0.48 | 0.42 | 0.37 |

As shown in Table 2, the area under the receiver operating characteristic curve (AUC) of the method for assessing acute kidney injury and the acute kidney injury assessment system of the present disclosure used to assess the occurrence of acute kidney injury of the testing dataset is 0.82, the accuracy thereof is 0.806, and the specificity thereof is 0.83. Further, the F-score of the method for assessing acute kidney injury and the acute kidney injury assessment system of the present disclosure used to assess the occurrence of acute kidney injury of the testing dataset is 0.53, the Matthews correlation coefficient (MCC) thereof is 0.43, and the kappa coefficient thereof is 0.42.

Reference is made to Table 3, which shows the verifying results of the occurrence of acute kidney injury obtained by the AKI assessing model of the method for assessing acute kidney injury and the acute kidney injury assessment system of the present disclosure based on the MutiLabel classification method.

TABLE 3

| | Training dataset | Testing dataset | Validation dataset |
|---|---|---|---|
| AUC (macro) | 0.877 | 0.802 | 0.799 |
| AUC (micro) | 0.969 | 0.958 | 0.958 |
| Accuracy | 0.889 | 0.865 | 0.867 |
| Precision (macro) | 0.891 | 0.504 | 0.672 |
| Recall (macro) | 0.497 | 0.445 | 0.446 |
| MCC | 0.53 | 0.41 | 0.4 |
| Kappa | 0.5 | 0.38 | 0.38 |

As shown in Table 3, the area under the receiver operating characteristic curve of the method for assessing acute kidney injury and the acute kidney injury assessment system of the present disclosure used to assess the occurrence of acute kidney injury of the testing dataset is 0.802, the accuracy thereof is 0.865, and the precision thereof is 0.504. Further, the recall rate of the method for assessing acute kidney injury and the acute kidney injury assessment system of the present disclosure used to assess the occurrence of acute kidney injury of the testing dataset is 0.455, the Matthews correlation coefficient thereof is 0.41, and the kappa coefficient thereof is 0.38.

### III. The Credibility of the Result of Kidney Care Classification Obtained by the Kidney Care Classifying Model of the Method for Assessing Acute Kidney Injury and the Acute Kidney Injury Assessment System of the Present Disclosure

In the present test, the kidney care classifying model is established based on the features, namely sociodemographic variables, comorbidities, baseline nephrotoxic agent exposure, baseline S-Cre and hemoglobin levels, and the risk matrix of CKO obtained therefrom is shown in Table 4.

TABLE 4

| Base eGFR | Last eGFR measurement | | |
|---|---|---|---|
| (ml/min/1.73 m²) | 60 ≤ eGFR | 45 ≤ eGFR < 60 | eGFR < 45 |
| Patient without | 1.00 | 4.24** | 8.99** |
| AKI | | (3.14-5.74) | (6.84-11.8) |
| Stable AKI patient | 22.31** | 48.53** | 56.30** |
| | (18.3-27.19) | (38.43-61.28) | (44.55-71.15) |
| Deteriorating AKI | 26.91** | 48.12** | 67.85** |
| patient | (18.43-39.29) | (36.11-64.12) | (54.24-84.87) |

**p < 0.0001, and the values shown in Table 4 are the risk multiples of the CKO.

As shown in Table 4, when the kidney care classifying model of the present disclosure is established based on sociodemographic variables, comorbidities, baseline nephrotoxic agent exposure, baseline S-Cre and hemoglobin levels, the kidney care classifying model of the present disclosure can effectively output the result of kidney care classification.

### IV. The Credibility of the Imputation by the Imputation Model of the Method for Assessing Acute Kidney Injury and the Acute Kidney Injury Assessment System of the Present Disclosure

The kidney function diagnostic dataset used in the imputation model of the present test is collected by China Medical University Hospital and includes 1,214,327 patients who have eGFR value with 7,271,195 exam records. After excluding the exam records which is not at an outpatient visit, the estimated glomerular filtration rate data is smaller than 15, the exam records without the formal identification, the exam records with duplicates, and the lab missing ratio thereof is >50%, there is a total of 229,891 patients and 627,169 exam records. Further, 389,600 exam records are served as the training dataset, 131,189 exam records are served as the testing dataset, and 129,690 exam records are served as the validation dataset.

In the present test, the imputation model is established by the XGBoost regression classifier with the Bayesian optimization process, and the credibility of the imputation model is evaluated by the regression method. There are two categories of feature set that are associated with the performance of the imputation model of the present disclosure applied in the imputation of the serum creatinine concentration data or the estimated glomerular filtration rate data, and the features in each of the two categories of feature set are shown in Table 5.

TABLE 5

| Category | Feature | Description | Example Value |
|---|---|---|---|
| Physiological | Age | patient's age | EX: 59.3 |
| data | | when visiting | |
| | Sex | biological sex | Male or Female |
| Biomedical | Blood_BUN | blood urine | EX: 20 mg/dL or |
| data | | nitrogen | 7.14 mmol/L |
| | | | (1 mg/dL = 0.357 |
| | | | mmol/L) |
| | Blood_P | serum phosphorus | EX: 3.5 mg/dL or |
| | | | 1.13 mmol/L |
| | | | (1 mg/dl = 0.323 |
| | | | mmol/L) |
| | Blood_Iron | serum iron(fe) | EX: 112 μg/dL or |
| | | | 20.05 μmol/L |
| | | | (1 μg/dL = 0.179 |
| | | | μmol/L) |
| | Urine_Protein | urine protein is | EX: +/−, +1, +2, +3 |
| | | reported using a | or +4 |
| | | semi-quantitative | |
| | | scale | |
| | uACR | urine | EX: 30 mg/g Cr or |
| | | albumin-to- | 3.39 mg/mmol Cr |
| | | creatinine | (1 mg/g Cr = 0.113 |
| | | ratio | mg/mmol Cr) |
| | Blood_HB | hemoglobin | EX: 15 g/dL or |
| | | | 150 g/L or 9.31 |
| | | | mmol/L |
| | | | (1 g/dL = 0.621 |
| | | | mmol/L) |
| | Blood_UA | serum uric acid | EX: 2.5 mg/dL or |
| | | | 148.7 μmol/L |
| | | | (1 mg/dl = 59.48 |
| | | | μmol/L) |
| | Blood_K | serum potassium | EX: 4.2 mmol/L or |
| | | | 4.2 mEq/L |
| | | | (1 mmol/L = 1 |
| | | | mEq/L) |
| | Blood_Albumin | serum albumin | EX: 4.1 g/dL or |
| | | | 41 g/L |
| | | | (1 g/dL = 10 g/L) |
| | Blood_Ca | serum calcium | EX: 9 mg/dL or |
| | | | 2.246 mmol/L |
| | | | (1 mg/dl = 0.2495 |
| | | | mmol/L ) |
| | Blood_Na | serum sodium | EX: 140 mmol/L or |
| | | | 140 mEq/L |
| | | | (1 mmol/L = 1 |
| | | | mEq/L) |
| | Blood TG | serum triglyceride | EX: 90 mg/dl or |
| | | | 1.017 mmol/L |
| | | | (1 mg/dl = 0.01130 |
| | | | mmol/L ) |
| | Blood_TCHO | serum cholesterol | EX: 160 mg/dl or |
| | | | 4.138 mmol/L |
| | | | (1 mg/dL = 0.02586 |
| | | | mmol/L) |

Further, the present test includes Example 1 and Example 2, wherein the imputation model of Example 1 and the imputation model of Example 2 are respectively established by the XGBoost regression classifier based on different feature composition. In detail, the imputation model of Example 1 is established based on all features shown in Table 5, and the imputation model of Example 2 is established based on the age, the sex, the value of urine protein, the value of hemoglobin, the value of serum uric acid and the value of serum triglyceride.

Further, the credibility of the imputation by the imputation model of the method for assessing acute kidney injury and the acute kidney injury assessment system of the present disclosure is evaluated by the regression method, and the results are shown in Table 6 and Table 7.

TABLE 6
Example 1

| | Training dataset | Testing dataset | Validation dataset |
|---|---|---|---|
| MAE | 8.782 | 9.944 | 9.898 |
| MSE | 128.909 | 172.44 | 167.399 |
| RMSE | 11.354 | 13.132 | 12.938 |

TABLE 7
Example 2

| | Training dataset | Testing dataset | Validation dataset |
|---|---|---|---|
| MAE | 12.94 | 13.17 | 13.14 |
| MSE | 284.1 | 297.6 | 296.1 |
| RMSE | 16.86 | 17.25 | 17.21 |

As shown in Table 6, the mean absolute error (MAE) of the imputation model of Example 1 used to analyze the testing dataset is 9.944, the mean square error (MSE) thereof is 172.44, and the root mean squared error (RMSE) thereof is 13.132. Further, the results obtained from the training dataset and the validation dataset are similar.

As shown in Table 7, the mean absolute error of the imputation model of Example 2 used to analyze the testing dataset is 13.17, the mean square error thereof is 297.6, and the root mean squared error thereof is 17.25. Further, the results obtained from the training dataset and the validation dataset are similar. The accuracy of the imputation model of Example 2 is slightly lower compared to Example 1, but the features used in Example 2 are more common in the clinical data, so that the imputation model of Example 2 is more versatile.

### V. Outcome Validation for Assessing the Acute Kidney Injury of the Method for Assessing Acute Kidney Injury and the Acute Kidney Injury Assessment System of the Present Disclosure

In the present test, the 18,947 patients of the validation dataset (“validation population” hereafter) of the kidney function diagnostic dataset are compared with the excluded 331,998 patients (including 146 patients who first visit the outpatient) of the kidney function diagnostic dataset (“unknown population” hereafter) so as to confirm whether the clinical prognosis of the predicted patient with AKI is worse than that of the patient without AKI obtained by the method for assessing acute kidney injury and the acute kidney injury assessment system of the present disclosure, and further compare the results obtained by the present disclosure with the conventional clinical standard for assessing the prognostic risk of AKI.

In detail, in the 18,947 patients of the validation population, the renal function values and the AKI statue of the patients are known, and the 331,998 patients of the unknown population have no renal function values that can be used to understand the AKI status thereof. Accordingly, the prognostic risks of AKI between the true patient with AKI and the true patient without AKI as well as the predicted patient with AKI and the predicted patient without AKI obtained by the present disclosure can be compared by the validation population, and the prognostic risks of AKI between the patient with AKI and the patient without AKI can be compared by the unknown population.

Further, in the outcomes associated with AKI, the clinical prognosis of AKI within one year is analyzed, wherein the clinical prognosis of AKI within one year includes the composite kidney outcomes of the patient with AKI, the all-cause mortality of the patient with AKI and the de novo chronic kidney disease (“CKD” hereafter) of the patient with AKI. The composite kidney outcomes include the occurrence of ESKD, the fluctuation value of eGFR decrease larger than 40% of the minimum estimated glomerular filtration rate data, or the fluctuation value of serum creatinine concentration increases larger than or equal to 2-fold of the minimum serum creatinine concentration data. The de novo CKD represents that the patient without the CKD (the minimum estimated glomerular filtration rate data<60 ml/min/1.73 m²) develops to the CKD within 3-12 months after the acute kidney injury assessing date (at least two minimum estimated glomerular filtration rate data<60 ml/min/1.73 m²) but not reach the level of ESKD.

Please refer to Table 8, which shows the analysis results of the outcome validation for assessing the acute kidney injury of the method for assessing acute kidney injury and the acute kidney injury assessment system of the present disclosure.

TABLE 8

| | | 1-year CKO | | 1-year Mortality | | 1-year De novo CKD | | |
|---|---|---|---|---|---|---|---|---|
| | | | OR | | OR | | | OR |
| Validation | Total | N | (95% | N | (95% | Total | N | (95% |
| population | N | (%) | CI) | (%) | CI) | N | (%) | CI) |
| | 18,947 | | | | | 16,122 | | |
| True AKI | 2,989 | 306 | 5.00 | 330 | 5.52 | 2,420 | 116 | 2.28 |
| | | (10.3) | (4.27, | (11.0) | (4.72, | | (4.8) | (1.83, |
| | | | 5.86) | | 6.45) | | | 2.84) |
| True | 15,958 | 356 | Ref | 351 | Ref | 13,702 | 296 | Ref |
| Non-AKI | | (2.2) | | (2.2) | | | (2.2) | |
| Predicted | 4,875 | 387 | 4.33 | 443 | 5.81 | 3,857 | 150 | 1.85 |
| AKI | | (7.9) | (3.69, | (9.1) | (4.95, | | (3.9) | (1.51, |
| | | | 5.07) | | 6.83) | | | 2.27) |
| Predicted | 14,072 | 275 | Ref | 238 | Ref | 12,265 | 262 | Ref |
| Non-AKI | | (1.9) | | (1.7) | | | (2.1) | |

| Unknown | Total | | | | | | | |
|---|---|---|---|---|---|---|---|---|
| population | N | 1-year CKO | | 1-year Mortality | | 1-year De novo CKD | | |
| | 331,998 | | | | | 311,935 | | |
| Predicted | 14,999 | 180 | 3.28 | 657 | 6.36 | 11,767 | 74 | 2.05 |
| AKI | | (1.20) | (2.80, | (4.38) | (5.82, | | (0.63) | (1.61, |
| | | | 3.84) | | 6.95) | | | 2.59) |
| Predicted | 316,999 | 1,169 | Ref | 2,267 | Ref | 300,168 | 926 | Ref |
| Non-AKI | | (0.37) | | (0.71) | | | (0.31) | |

As shown in Table 8, the clinical prognosis risks of AKI of both the true patient with AKI and the predicted patient with AKI are significantly higher than those of the true patient without AKI, and the difference in risk between the true patient with AKI and the predicted patient with AKI is not significant, wherein the risk of CKO is about 4-5 times, the risk of mortality is about 6 times, and the risk of the de novo CKD is about 2 times. Further, in the unknown population, the patients are healthier, and thus the predicted occurrence of AKI rate and the clinical prognosis risks thereof will be much lower. However, the clinical prognosis risk of AKI of the predicted patient with AKI is still significantly higher than that of the patient without AKI, wherein with the risk of CKO is approximately 3 times, the risk of mortality is approximately 6 times, and the risk of de novo CKD is approximately 2 times.

Therefore, there is a correlation between the predicted patient with AKI and the clinical prognosis risks of AKI, and thus the method for assessing acute kidney injury and the acute kidney injury assessment system of the present disclosure have the application potentials in the clinical.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

1. A method for assessing acute kidney injury (AKI), comprising:
obtaining a kidney function diagnostic dataset of a subject by a processor, wherein the kidney function diagnostic dataset comprises at least one serum creatinine concentration data or at least one estimated glomerular filtration rate (eGFR) data of the subject, and a recording date of the at least one serum creatinine concentration data and a recording date of each of the at least one estimated glomerular filtration rate data are respectively on 0 to 180 days before an acute kidney injury assessing date of the subject;
performing a preprocessing step by the processor, wherein a changing degree over a time period of the at least one serum creatinine concentration data or a changing degree over a time period of the at least one estimated glomerular filtration rate data is calculated by the processor so as to obtain at least one fluctuation value of serum creatinine concentration or at least one fluctuation value of eGFR; and
performing a first analyzing step by the processor, wherein the at least one fluctuation value of serum creatinine concentration or the at least one fluctuation value of eGFR is analyzed by an AKI assessing model of the processor so as to define the subject as a patient with AKI or a patient without AKI.

2. The method for assessing acute kidney injury of claim 1, wherein the patient with AKI has the at least one fluctuation value of serum creatinine concentration larger than or equal to 50% or has the at least one fluctuation value of eGFR larger than or equal to 35%.

3. The method for assessing acute kidney injury of claim 1, wherein the at least one serum creatinine concentration data comprises a maximum serum creatinine concentration data and a minimum serum creatinine concentration data, the at least one estimated glomerular filtration rate data comprises a maximum estimated glomerular filtration rate data and a minimum estimated glomerular filtration rate data, the at least one fluctuation value of serum creatinine concentration is calculated based on the maximum serum creatinine concentration data and the minimum serum creatinine concentration data, and the at least one fluctuation value of eGFR is calculated based on the maximum estimated glomerular filtration rate data and the minimum estimated glomerular filtration rate data.

4. The method for assessing acute kidney injury of claim 1, wherein the AKI assessing model is established by a Cox regression calculating classifier.

5. The method for assessing acute kidney injury of claim 1, wherein the kidney function diagnostic dataset further comprises a base serum creatinine concentration data, the at least one serum creatinine concentration data comprises a first serum creatinine concentration data, a recording date of the base serum creatinine concentration data is the acute kidney injury assessing date, and a recording date of the first serum creatinine concentration data is the closest to the acute kidney injury assessing date in the at least one serum creatinine concentration data;
wherein the method for assessing acute kidney injury further comprises:
performing a second analyzing step by the processor, wherein the base serum creatinine concentration data and the first serum creatinine concentration data are calculated by the processor so as to obtain a difference value of serum creatinine concentration, and the difference value of serum creatinine concentration is analyzed by the AKI assessing model so as to assess that the patient with AKI is a deteriorating AKI patient or a stable AKI patient.

6. The method for assessing acute kidney injury of claim 5, wherein the deteriorating AKI patient has the difference value of serum creatinine concentration larger than or equal to 0.3 mg/dL, and the stable AKI patient has the difference value of serum creatinine concentration smaller than 0.3 mg/dL.

7. The method for assessing acute kidney injury of claim 1, wherein the kidney function diagnostic dataset further comprises a base estimated glomerular filtration rate data, and a recording date of the base estimated glomerular filtration rate data is the acute kidney injury assessing date;
wherein the method for assessing acute kidney injury further comprises:
performing a kidney care classifying step by the processor, wherein the base estimated glomerular filtration rate data is analyzed by the AKI assessing model so as to obtain a result of kidney care classification.

8. The method for assessing acute kidney injury of claim 1, further comprising:
performing a nephrotoxic drug screening step by the processor, wherein the kidney function diagnostic dataset further comprises a drug utilization data, and the drug utilization data is analyzed by the AKI assessing model in the nephrotoxic drug screening step so as to output a screening result of nephrotoxic drug usage.

9. The method for assessing acute kidney injury of claim 1, further comprising:
performing an imputation step by the processor, wherein the kidney function diagnostic dataset further comprises a physiological data and a biomedical data of the subject, and the physiological data and the biomedical data of the subject are analyzed by an imputation model of the processor so as to obtain the at least one serum creatinine concentration data or the at least one estimated glomerular filtration rate data.

10. The method for assessing acute kidney injury of claim 9, wherein the imputation model is established by an XGBoost regression classifier.

11. The method for assessing acute kidney injury of claim 10, wherein the physiological data and the biomedical data are respectively processed with a Bayesian optimization process by the imputation model so as to obtain a feature set of the imputation model.

12. The method for assessing acute kidney injury of claim 10, wherein the physiological data comprises an age and a sex of the subject, and the biomedical data comprises a value of blood urine nitrogen, a value of serum phosphorus, a value of serum iron, a value of urine protein, a value of urine albumin-to-creatinine ratio, a value of hemoglobin, a value of serum uric acid, a value of serum potassium, a value of serum albumin, a value of serum calcium, a value of serum sodium, a value of serum triglyceride and a value of serum cholesterol.

13. An acute kidney injury (AKI) assessment system, comprising:
a memory for storing a kidney function diagnostic dataset of a subject, wherein the kidney function diagnostic dataset comprises at least one serum creatinine concentration data or at least one estimated glomerular filtration rate (eGFR) data, and the at least one serum creatinine concentration data and the at least one estimated glomerular filtration rate data are respectively recorded on 0 to 180 days before an acute kidney injury assessing date; and
a processor signally connected to the memory, and the processor comprising:
a preprocessing model for calculating a changing degree over a time period of the at least one serum creatinine concentration data or a changing degree over a time period of the at least one estimated glomerular filtration rate data so as to obtain at least one fluctuation value of serum creatinine concentration or at least one fluctuation value of eGFR; and
a first analyzing model signally connected to the preprocessing model, wherein the first analyzing model comprises an AKI assessing model, and the at least one fluctuation value of serum creatinine concentration or the at least one fluctuation value of eGFR is analyzed by the AKI assessing model so as to define the subject as a patient with AKI or a patient without AKI.

14. The acute kidney injury assessment system of claim 13, wherein the patient with AKI has the at least one fluctuation value of serum creatinine concentration larger than or equal to 50% or has the at least one fluctuation value of eGFR larger than or equal to 35%.

15. The acute kidney injury assessment system of claim 13, wherein the at least one serum creatinine concentration data comprises a maximum serum creatinine concentration data and a minimum serum creatinine concentration data, the at least one estimated glomerular filtration rate data comprises a maximum estimated glomerular filtration rate data and a minimum estimated glomerular filtration rate data, the at least one fluctuation value of serum creatinine concentration is calculated based on the maximum serum creatinine concentration data and the minimum serum creatinine concentration data, and the at least one fluctuation value of eGFR is calculated based on the maximum estimated glomerular filtration rate data and the minimum estimated glomerular filtration rate data.

16. The acute kidney injury assessment system of claim 13, wherein the AKI assessing model is established by a Cox regression calculating classifier.

17. The acute kidney injury assessment system of claim 13, wherein the kidney function diagnostic dataset further comprises a base serum creatinine concentration data, the at least one serum creatinine concentration data comprises a first serum creatinine concentration data, a recording date of the base serum creatinine concentration data is the acute kidney injury assessing date, and a recording date of the first serum creatinine concentration data is the closest to the acute kidney injury assessing date in the at least one serum creatinine concentration data;
wherein the processor further comprises:
a second analyzing model signally connected to the first analyzing model, wherein the base serum creatinine concentration data and the first serum creatinine concentration data are calculated by the second analyzing model so as to obtain a difference value of serum creatinine concentration, and the difference value of serum creatinine concentration is analyzed by the AKI assessing model so as to assess that the patient with AKI is a deteriorating AKI patient or a stable AKI patient.

18. The acute kidney injury assessment system of claim 17, wherein the deteriorating AKI patient has the difference value of serum creatinine concentration larger than or equal to 0.3 mg/dL, and the stable AKI patient has the difference value of serum creatinine concentration smaller than 0.3 mg/dL.

19. The acute kidney injury assessment system of claim 13, wherein the kidney function diagnostic dataset further comprises a base estimated glomerular filtration rate data, and a recording date of the base estimated glomerular filtration rate data is the acute kidney injury assessing date;
wherein the processor further comprises:
a kidney care classifying model signally connected to the first analyzing model, wherein the kidney care classifying model is for analyzing the base estimated glomerular filtration rate data by the AKI assessing model so as to obtain a result of kidney care classification.

20. The acute kidney injury assessment system of claim 13, wherein the processor further comprises:
a nephrotoxic drug screening model signally connected to the first analyzing model, wherein the kidney function diagnostic dataset further comprises a drug utilization data, and the nephrotoxic drug screening model is for analyzing the drug utilization data so as to output a screening result of nephrotoxic drug usage.

21. The acute kidney injury assessment system of claim 13, wherein the processor further comprises an imputation model, the kidney function diagnostic dataset further comprises a physiological data and a biomedical data of the subject, and the physiological data and the biomedical data of the subject are analyzed by the imputation model of the processor so as to obtain the at least one serum creatinine concentration data or the at least one estimated glomerular filtration rate data.

22. The acute kidney injury assessment system of claim 21, wherein the imputation model is established by an XGBoost regression classifier.

23. The acute kidney injury assessment system of claim 22, wherein the physiological data and the biomedical data are respectively processed with a Bayesian optimization process by the imputation model so as to obtain a feature set of the imputation model.

24. The acute kidney injury assessment system of claim 21, wherein the physiological data comprises an age and a sex of the subject, and the biomedical data comprises a value of blood urine nitrogen, a value of serum phosphorus, a value of serum iron, a value of urine protein, a value of urine albumin-to-creatinine ratio, a value of hemoglobin, a value of serum uric acid, a value of serum potassium, a value of serum albumin, a value of serum calcium, a value of serum sodium, a value of serum triglyceride and a value of serum cholesterol.
